(19) United States
(12) Patent Application Publication
(10) Pub. No.: US 2025/0288469 A1
(43) Pub. Date: Sep. 18, 2025

(54) ABSORBENT ARTICLE WITH EMBOSSED SHAPING ELEMENTS

(71) Applicant: Essity Hygiene and Health Aktiebolag, Göteborg (SE)

(72) Inventors: Carin HÅKANSSON, Göteborg (SE); Angelica BURVALL, Göteborg (SE); Pia KALENTUN, Göteborg (SE); Moa HOLMSTRÖM, Malmö (SE)

(73) Assignee: Essity Hygiene and Health Aktiebolag, Göteborg (SE)

(21) Appl. No.: 18/860,862

(22) PCT Filed: Jun. 22, 2022
(86) PCT No.: PCT/EP2022/066951

§ 371 Date: Oct. 28, 2024

(51) Int. Cl. A61F 13/475; A61F 13/47; A61F 13/511; A61F 13/53

(52) U.S. Cl. CPC A61F 13/4756; A61F 13/51104; A61F 13/53; A61F 2013/4708

(57) ABSTRACT

An absorbent article for a female user in the form of a sanitary napkin, incontinence pad or liner as disclosed herein has a fluid permeable topsheet, a backsheet and an absorbent core. The absorbent core includes a first absorbent structure located between the topsheet and a second absorbent structure, and the second absorbent structure is located between the first absorbent structure and the backsheet. The second absorbent structure includes a central embossed shaping element, including an I-shaped portion extending along at least part of a longitudinal centreline of the absorbent core. The absorbent article also includes an outer embossed shaping element provided in at least either of the topsheet or the first absorbent structure. The outer embossed shaping element includes a first outer embossed shaping portion and a second outer embossed shaping portion, each extending in the longitudinal direction and on a respective side of the central embossed shaping element.

### TECHNICAL FIELD

The present disclosure pertains to an absorbent article for a female user in the form of a sanitary napkin, incontinence pad or liner having an absorbent core provided with embossed shaping elements to promote a good fit and leakage security.

### BACKGROUND OF THE INVENTION

Absorbent articles for female user such as sanitary napkins, incontinence pads or liners to which this disclosure relates are typically intended to absorb body liquids, such as urine, menses and other body fluids. Such articles may be absorbent pads for heavier bladder leakage, thinner liners for light bladder leakage and other body discharges, or sanitary napkins for menses.

Absorbent articles for female users are generally provided with an absorbent core to receive and retain body liquids. For incontinence pads intended for heavier bladder leakage to function efficiently, the absorbent core must quickly acquire body liquids into the structure from the point of application and subsequently distribute the body liquids within and throughout the absorbent core to provide maximum leakage security. It is also desirable that such absorbent articles are comfortable and have a good fit, i.e., to be sized and configured to fit the limited space available in the crotch portion of the underwear while providing the wearer with a feeling of security. Additionally, there is desire that such articles are discreet, thin, and not visible even if the user wears tight fitting, which may be seen as being in contradiction with the above objects.

It is furthermore of importance that the absorbent article for female user, gives the user a visual perception of leakage security and good fit, enabling the user to feel secure and thereby enhancing the well-being of the user.

In view of the above, there is a need for absorbent article for female user, which does not leak, has good fit and is discreet to wear.

### SUMMARY OF THE INVENTION

One or more of the above objects is achieved with an absorbent article for female user according to claim 1. Further advantages and advantageous features of the invention are disclosed in the following description and in the dependent claims.

An absorbent article for female user as disclosed herein has a first and a second longitudinal side edge and a front and a rear end edge. The absorbent article for female user comprises a fluid permeable topsheet, a backsheet and an absorbent core being located between the topsheet and the backsheet. The absorbent article extends in a longitudinal direction L, a transverse direction T and a thickness direction and has a longitudinal centreline Lc. The absorbent core is divided in a front half and a rear half. The absorbent article comprising a first absorbent structure and a second absorbent structure. The first absorbent structure is located between the topsheet and the second absorbent structure, and the second absorbent structure is located between the first absorbent structure and the backsheet. The second absorbent structure comprising a central embossed shaping element. The central embossed shaping element comprising an I-shaped portion extending along at least a part of the longitudinal centreline Lc of the absorbent core. The absorbent article also comprises an outer embossed shaping element provided in the topsheet and/or the first absorbent structure. The outer embossed shaping element comprising a first outer embossed shaping portion and a second outer embossed shaping portion, each extending in the longitudinal direction and on a respective side of the central embossed shaping element. The first outer embossed shaping portion is, in the transversal direction, provided on a respective side of the central embossed shaping element. The outer embossed shaping element is provided in at least either of the topsheet or the first absorbent structure.

The central embossed shaping element is provided in the second absorbent structure and the outer embossed shaping element is provided in either the topsheet or the first absorbent structure, or alternatively the outer embossed shaping element may be provided in both the topsheet and the first absorbent structure. The first absorbent structure may comprise a fluid control layer and/or a first absorbent layer. The outer embossed shaping element may be provided in the topsheet, the fluid control layer and/or the first absorbent layer. The absorbent article comprises an outer embossed shaping element provided in at least one layer which is located above the layer and this construction promotes a good flexing and shaping of the article which is also predictable. The first absorbent structure may comprise a fluid control layer and a first absorbent layer. The fluid control layer may be located between the topsheet and the first absorbent layer, or alternatively, between the first absorbent layer and the second absorbent structure. According to this embodiment, the outer embossed shaping element is provided in the topsheet and the layer closest to the topsheet of either the fluid control layer or the first absorbent layer.

According to one embodiment comprises the first absorbent structure a fluid control layer and a first absorbent layer, wherein the outer embossed shaping element is provided in the topsheet, the fluid control layer and the first absorbent layer.

The second absorbent structure comprises at least a one absorbent layer, a second absorbent layer, but may also comprise more than one layers. The central embossed element is provided in the second absorbent structure, at least in one absorbent layer in the second absorbent structure.

The first absorbent structure may have a greater size, in the plane of the article, than the second absorbent structure. If the first absorbent structure comprising more than one layer, at least one layer in the first absorbent structure may have a smaller size in the plane of the article than the second absorbent structure, for example may the fluid control layer be of less size than the second absorbent structure. The fluid control layer may for example have shorter length than the first absorbent layer. The first and the second embossed shaping portions of the outer embossed shaping element each extend rearwardly of a rear edge of the fluid control layer and may comprise a respective line rupture of the first and second embossed shaping portions coinciding with the rear edge of the fluid control layer.

The first absorbent structure may be larger than the second absorbent structure, for example may a first absorbent layer in the first absorbent structure be larger than the second absorbent structure. However, the second absorbent structure may alternatively be larger than the first absorbent structure.

Each of the first and second outer embossed shaping portions may be formed by a continuous embossed line, or may alternatively be formed by two or more continuous embossed lines. Alternatively, the outer embossed shaping element may be formed by a continuous or discontinuous embossment pattern extending in the longitudinal direction L and on a respective side of the central embossed shaping element.

The outer embossed shaping element may also comprise print pattern to further visualize the outer embossed shaping element.

The central embossed shaping element may extend at least along part of the rear half of the absorbent core and/or at least along part of the front half of the absorbent core.

The first and the second embossed elements of the outer embossed element may converge in the direction from the front end edge of the absorbent article towards the rear end edge of the absorbent article. The transversal distance between the first and second embossed shaping portions closest to the front end edge of the article may be 30-60 mm and the transversal distance between the first and second embossed shaping portions of the outer embossed shaping element closest to the rear end edge of the article may be 0.5-20 mm. A greater distance between the first and second embossed shaping portions of the outer embossed shaping element in the front half promotes the absorbent core, during use of the article to flex in the front half of the absorbent article and to adopt a bowl like shape in the inlet zone in the front half of the absorbent core.

The outer embossed shaping element may extend up to or past a rear end point of the central embossed shaping element and/or may extend up to or past a front end point of the central embossed shaping element.

The outer embossed shaping element may further comprise an arc-shaped embossed front portion arranged frontwards of the central embossed shaping element. The arc-shaped embossed front portion may together with the central shaping element promote an enhanced and predictable flexing and adopt a bowl like shape around the inlet zone in the front half of the article.

The transversal distance between the first and the second outer embossed shaping portions in the rear half may vary less than 12%, optionally less than 10%, or less than 5%.

The transversal width of the absorbent core along the rear half may vary less than 12%, optionally less than 10%, or less than 5%.

The outer embossed shaping element may comprise a third outer embossed shaping portion and a fourth outer embossed shaping portion, optionally provided in the topsheet and the first absorbent structure, the third outer embossed shaping portion being provided outwards with respect to the first outer embossed shaping portion of the outer embossed shaping element and the fourth lateral embossed shaping portion being provided outwards with respect to the second outer embossed shaping portion of the outer embossed shaping element. The third and the fourth outer embossed portions being arranged on a respective side of the I-shaped portion of the central embossed shaping element. The third and the fourth outer embossed lines may be arranged rearwards of the inlet zone and on a respective side of the I-shaped portion. The third and the fourth outer embossed lines may flatten the zone of the article being arranged in the crotch zone of the user and rearwardly with respect to the inlet zone.

The second absorbent structure may comprise superabsorbent polymers in an amount of from 30 wt. %, optionally from 35 wt % or optionally from 40 wt. % based on the total second absorbent layer, such as within the range of from 30 wt %, 35 wt % or 40 wt. % to 80 wt. %. Due to a higher content of superabsorbent polymers in such absorbent core, the core structure may be perceived as less flexible and less prone to shape in response to the user body. However, such absorbent article may become difficult to shape in a predictable manner. The central embossed shaping element and the outer embossed shaping element however enables an improved flexing and shaping of such absorbent article.

The second absorbent structure may have a higher density than the first absorbent structure. The second absorbent structure may have a higher density than the fluid control layer, the second absorbent may additionally or alternatively have a higher density than the first absorbent layer. The second absorbent layer may have at least 50%, such as 75% or 100% higher density than each of the fluid control layer and the first absorbent layer. The first absorbent layer may have a higher density than the fluid control layer. The first absorbent layer may have at least 25% higher density than the fluid control layer.

The second absorbent structure may comprise a first side cut along the first longitudinal side edge and a second side cut along the second longitudinal side edge. The first and the second side cuts may be arranged in the front half of the absorbent core. A ratio of a width W₁₃ at the narrowest section of the second absorbent structure at the first and the second side cuts and a width of the absorbent article W₁ at the same location may be from 0.5:1 to 0.8:1. This has been found providing an enhanced cooperation with the central embossing shaping elements for providing the pad in a desired and predictable manner.

The first and the second embossed parts of the outer embossed element may extend on a respective side of a longitudinal centerline of the pad in a rear third of the absorbent core, and a distance between the first and the second embossed parts may be within the range of from 0.5-20 mm, optionally within the range of from 0.5 to 12 mm or optionally within the range of from 5-12 mm. This promotes an improved flexing of the pad along the I-shaped portion of the central embossed shaping element. This has been found to cooperate with the central embossed shaping element and improve the prediction of the shaping in the rear part of the article. Since the absorbent article may be a multilayer pad with a fluid control layer arranged between the topsheet and the first absorbent layer, the predictability of the shaping of the pad in the rear part may improve when first and second embossed lines of the outer embossed element extend on a respective lateral side of the I-shaped portion as well as on a respective lateral side of the longitudinal centerline Lc.

The absorbent core has a front part, a rear part and an intermediate part located between the front part and the rear part, and the widest transversal width of the absorbent core may be in the front part. A transition width (W₈) between the front part and the intermediate part of the absorbent core may be narrower than the transversal width of the rest of the absorbent core. The second absorbent structure may have a narrower transversal width W₁₃ between the front part and the intermediate part of the absorbent core than the transversal width of the absorbent core W₈. The transversal width of the second absorbent structure, W₁₃, may be narrower than the transversal width of the first absorbent layer between the front part and the intermediate part of the absorbent core. The front part, the rear part and the intermediate part of the absorbent core may be of substantially equal length. The central embossed shaping element may be extending in the front part and in the intermediate part of the absorbent core. The outer embossed shaping element may be extending in the front part and in the intermediate part of the absorbent core. The outer embossed shaping element may also extend in part of the rear part of the absorbent core, and according to this embodiment has the outer embossed shaping element a greater extension in the longitudinal direction in the rear half than the central embossed shaping element. The outer embossed shaping element may have a greater extension in the longitudinal direction, also in the front part, towards the front end, than the central embossed shaping element.

The central embossed shaping element and/or the outer embossed shaping element may comprise continuous or discontinuous embossment portions.

### BRIEF DESCRIPTION OF THE DRAWINGS

The present invention will be further explained hereinafter by means of non-limiting examples and with reference to the appended drawings wherein:

FIG. 1 shows a top plan view of an incontinence pad as disclosed herein;

FIG. 2 shows an exploded view of the incontinence pad in FIG. 1; and

FIG. 3 shows the incontinence pad from FIG. 1, flexed and shaped as during use.

### DETAILED DESCRIPTION

The invention will be described more closely below by reference to an exemplary embodiment. The invention may however be embodied in many different forms and should not be construed as limited to the embodiments set forth in the drawings and the description thereto.

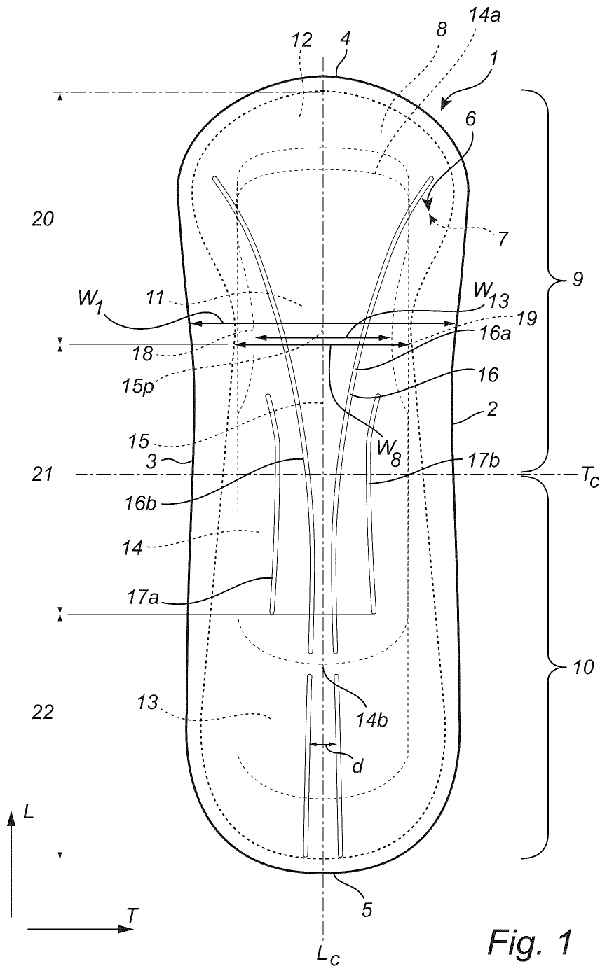

FIGS. 1 to 3 show an incontinence pad 1 according to the present disclosure. FIG. 1 schematically shows the incontinence pad 1 seen from the side that is intended to be facing towards a wearer's body when the pad 1 is being worn.

The incontinence pad 1 has a first and a second longitudinal side edge 2,3 and a front and a rear end edge 4,5. The pad 1 comprises a fluid permeable topsheet 6, a backsheet 7 and an absorbent core 8 being located between the topsheet 6 and the backsheet 7.

The incontinence pad 1 extends in a longitudinal direction L and a transverse direction T and the absorbent core 8 is divided in a front half 9 and a rear half 10 and has an inlet zone in the front half 9.

The absorbent core 8 comprises a first absorbent structure 11 having a first absorbent layer 12 and a fluid control layer 14, and a second absorbent structure 13. The first absorbent layer 12 being arranged between the fluid control layer 14 and the second absorbent structure 13 and the fluid control layer 14 being arranged between the topsheet 6 and the first absorbent layer 12.

The topsheet 6 may include or consist of fibrous nonwoven layer(s) being spunbonded, meltblown, carded, hydroentangled, wetlaid. Suitable nonwoven materials can be composed of natural fibers, such as woodpulp or cotton fibers, synthetic thermoplastic fibers, such as polyolefins, polyesters, polyamides and blends and combinations thereof or from mixtures of natural and synthetic fibers. The topsheet 6 may consist of non-absorbent fibers, such as thermoplastic fibers. The materials suited as topsheet material should be soft and non-irritating to the skin and be readily penetrated by body fluid, such as menstrual fluid and urine.

The backsheet 7 may comprise or consist of a thin plastic film, e.g., a polyethylene or polypropylene film, a nonwoven material coated with a liquid impervious material, a hydrophobic nonwoven, which resist liquid penetration. Laminates of plastic films and nonwoven materials may also be used. The backsheet material can be breathable to allow vapor to escape from the absorbent structure, while still preventing liquids from passing through the backsheet material.

The absorbent core 8 constitutes the absorbent structure of the article which acquires, distributes and stores bodily fluids and comprises the fluid control layer, the first absorbent layer and the second absorbent layer. The first and second absorbent layers may be of any conventional kind. Examples of commonly occurring absorbent materials are cellulosic fluff pulp, tissue, highly absorbent polymers (so called superabsorbents), absorbent foam materials, absorbent nonwoven materials or the like. It is common to combine cellulosic fluff pulp with superabsorbent polymers in an absorbent core. Superabsorbent polymers are water-swellable, water-insoluble organic or inorganic materials capable of absorbing at least about 20 times their own weight of an aqueous solution containing 0.9 weight percent of sodium chloride. Organic materials suitable for use as a superabsorbent material can include natural materials such as polysaccharides, polypeptides and the like, as well as synthetic materials such as synthetic hydrogel polymers. Such hydrogel polymers include, for example, alkali metal salts of polyacrylic acids, polyacrylamides, polyvinyl alcohol, polyacrylates, polyacrylamides, polyvinyl pyridines, and the like. Other suitable polymers include hydrolyzed acrylonitrile grafted starch, acrylic acid grafted starch, and isobutylene maleic anhydride copolymers and mixtures thereof. The hydrogel polymers are preferably lightly cross-linked to render the material substantially water insoluble. Preferred superabsorbent materials are further surface cross-linked so that the outer surface or shell of the superabsorbent particle, fibre, flake, sphere, etc. possesses a higher crosslink density than the inner portion of the superabsorbent. The superabsorbent materials may be in any form suitable for use in absorbent composites including particles, fibres, flakes, spheres, and the like. A high absorption capacity is provided using high amounts of superabsorbent material. Thin absorbent cores which are common in for example sanitary napkins, baby diapers and incontinence guards, often comprise a compressed, mixed or layered structure of cellulosic fluff pulp and superabsorbent polymers. The size and absorbent capacity of the absorbent core may be varied to suit different product types, such as smaller incontinence pads for day use or larger and longer ones for night-time use.

The first absorbent layer 12 may more specifically be an airlaid nonwoven layer or a matformed absorbent layer.

The second absorbent structure 13 may comprise superabsorbent polymers in an amount of 40 wt. % or more, such as 50 wt. % or more, optionally within the range of from 40% to 70%. The superabsorbent polymer may for example be mixed with cellulosic fluff pulp. The second absorbent structure 13 is optionally wrapped in a core wrap.

The fluid control layer 14 arranged between the topsheet 6 and the first absorbent layer 12 is adapted to quickly receive and temporarily store discharged liquid before it is absorbed by the first absorbent layer 12 and the second absorbent structure 13. The fluid control layer 14 may be a fibrous layer comprising or consisting of synthetic fibers and may for example be airlaid nonwoven, spunlace nonwoven or high loft nonwoven.

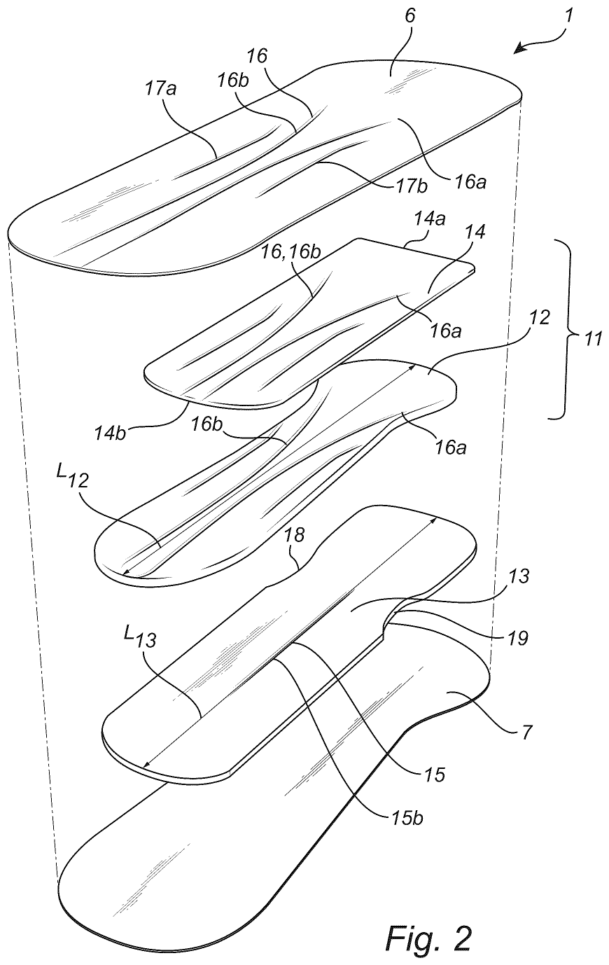

A length of the fluid control layer 14, as illustrated in FIG. 2, is within the range of from 30% to 70% of a length L₁₃ of the second absorbent layer 13 and the fluid control layer 14 extends no further than over 75% of the first absorbent layer 12.

The second absorbent layer 13 is provided with a central embossed shaping element 15 in the form of an I-shaped element 15b provided at least partly in the front half 9 and partly in the rear half 10 of the absorbent core. The I-shaped portion 15b may be a continuous or a discontinuous I-shaped portion. The central embossed shaping element 15 may constitute of a continuous embossing or by discontinuous elements, such as dots, forming the central embossed shaping element 15.

The incontinence pad 1 comprises an outer embossed shaping element 16 provided in the topsheet 6, the fluid control layer 14 and the first absorbent layer 12. The outer embossed shaping element 16 comprising a first embossed line 16a and a second embossed line 16b, each extending in the longitudinal direction L and on a respective side of the central embossed shaping element 15. The first and the second embossed lines 16a, 16b of the outer embossed shaping element 16 converge towards the rear end edge 5 of the pad 1.

In FIGS. 1-3, the first and second embossed lines 16a, 16b extend up to a rear edge 14b of the fluid control layer 14, such as with a distance within the range of from 2 to 10 mm from the rear edge 14b of the fluid control layer 14. At the region coinciding with the rear edge 14b, as seen from above, there is a respective line rupture, i.e., an area free from embossing, in the first and second embossed lines 16a, 16b. The first and second embossed lines 16a, 16b extend rearwards the rear edge 14b of the fluid control layer 14, with the first and second embossed lines 16a, 16b only being provided in the topsheet layer 6 rearwards the rear edge 14b of the fluid control layer 14. The first and second embossed lines 16a, 16b are provided with a distance d to each other in the rear part of the absorbent core 8 as measured in the transverse direction T, may be within the range of from 0.5-20 mm, optionally within the range of from 0.5 to 12 mm or optionally within the range of from 5-12 mm. This promotes an improved flexing of the pad along the I-shaped portion 15b of the central embossed shaping element 15.

The outer embossed element 16 furthermore comprises a third outer embossed line 16d and a fourth outer embossed line 16e provided in the topsheet 6, the fluid control layer 14 and the first absorbent layer 12. The third outer embossed line 16d is provided outwards with respect to the first embossed line 16a of the outer embossed element 16 and the fourth outer embossed line 16e being provided outwards with respect to the second outer embossed line 16b of the outer embossed element 16. The third and the fourth outer embossed lines 16d, 16e are arranged rearwards of the front part 20 of the absorbent core 8 and on a respective side of the I-shaped portion 15b.

The second absorbent layer 13 may comprise superabsorbent polymers in an amount of from 30 wt. %, optionally in an amount of from 40 wt. % to 80 wt. %. The second absorbent structure 13 in the incontinence pad 1 is one absorbent layer, the second absorbent layer 13, but the second absorbent structure may also comprise more than one absorbent layer. The second absorbent structure 13 comprises a first side cut 19 along the first longitudinal side edge 2 and a second side cut 20 along the second longitudinal side edge 3.

A ratio of a width W₁₃ at the narrowest section of the second absorbent structure 13 at the first and the second side cuts 19,20 and a width W₁ of the incontinence pad 1 at the same location is from 0.5:1 to 0.8:1.

As illustrated in FIGS. 1 and 2, the width of the second absorbent structure 13 is relatively constant, except at the first and second side cuts 19,20. In FIG. 1-2 is the second absorbent structure 13 larger than the fluid control layer 14 and the first absorbent layer 12. However, the first absorbent structure may be larger than the second absorbent structure, for example may a first layer in the first absorbent structure be larger than the second absorbent structure 13.

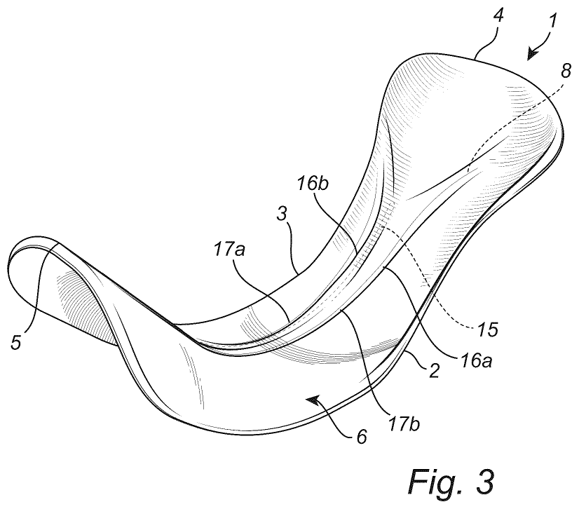

FIG. 3 illustrates the incontinence pad 1 from FIGS. 1 and 2 when being flexed and shaped as during use. The disclosed configuration of the incontinence pad 1 with a combination of a central embossed shaping element 15 in a second absorbent structure 13 below a first absorbent structure with an outer embossed shaping element enables an improved and predictable shaping during use of the incontinence pad 1. During use, the incontinence pad 1 closely adopt to the female anatomy and allows the front half of the article to fit over and conform to the flattened forward portion of the female genitalia and to form a front bowl shape for acquiring the body liquids, while the rear half of the article aligns with and fit comfortable into the crease-like regions of the buttocks and perineum.

### Thickness Measurement

To determine the density of the first and the second absorbent structure is the thickness determined by means of a measuring foot with affixed load of 0.5 k Pa. The foot is lowered onto the absorbent structure at a rate of 13 mm/s. The thickness is read off at the digital thickness gauge/tester after 5 seconds when the measuring foot has touched the surface of the absorbent structure.

1. An absorbent article for female user in the form of a sanitary napkin, incontinence pad or liner having a first and a second longitudinal side edge and a front and a rear end edge, and comprising a fluid permeable topsheet, a backsheet and an absorbent core being located between the topsheet and the backsheet, the absorbent article is extending in a longitudinal direction, a transverse direction and a thickness direction and has a longitudinal centreline, the absorbent core being divided in a front half and a rear half and comprising a first absorbent structure and a second absorbent structure, the first absorbent structure is located between the topsheet and the second absorbent structure, and the second absorbent structure is located between the first absorbent structure and the backsheet, wherein the second absorbent structure comprises a central embossed shaping element, the central embossed shaping element comprising an I-shaped portion extending along at least part of the longitudinal centreline of the absorbent core, the absorbent article comprising also an outer embossed shaping element provided in the topsheet and/or the first absorbent structure, the outer embossed shaping element comprising a first outer embossed shaping portion and a second outer embossed shaping portion, each extending in the longitudinal direction and on a respective side of the central embossed shaping element.

2. The absorbent article according to claim 1, wherein the first absorbent structure comprises a fluid control layer and a first absorbent layer.

3. The absorbent article according to claim 2 wherein the outer embossed shaping element is provided in the topsheet, the fluid control layer and the first absorbent layer.

4. The absorbent article according to previous-claim 1, wherein the fluid control layer has a shorter length than the first absorbent layer and the first and the second embossed parts of the outer embossed shaping element each extend rearwardly of a rear edge of the fluid control layer and comprises a respective line rupture of the first and second embossed parts coinciding with the rear edge of the fluid control layer.

5. The absorbent article according to claim 1, wherein the central embossed shaping element extends along at least part of the rear half of the absorbent core and along at least part of the front half of the absorbent core.

6. The absorbent article according to claim 1, wherein the first and the second outer embossed shaping portions of the outer embossed shaping element converges in the direction from the front end edge of the absorbent article towards the rear end edge of the absorbent article.

7. The absorbent article according to claim 1, wherein the transversal distance between the first and the second outer embossed shaping portions closest to the front end edge of the article is 30-60 mm and the transversal distance between the first and the second embossed shaping portions closest to the rear end edge of the article is 0.5-20 mm.

8. The absorbent article according to claim 1, wherein the outer embossed shaping element extends up to or past a rear end point of the central embossed shaping element or extends up to or past a front end point of the central embossed shaping element.

9. The absorbent article according to claim 1, wherein the transversal distance between the first and the second outer embossed shaping portions in the rear half varies less than 12%.

10. The absorbent article according to claim 1, wherein the transversal width of the absorbent core along the rear half varies less than 12%, optionally less than 10%, or less than 5%.

11. The absorbent article according to claim 1, wherein the outer embossed shaping element comprises a third outer embossed shaping portion and a fourth outer embossed shaping portion provided in the topsheet and the first absorbent structure, the third outer embossed shaping portion being provided outwards with respect to the first outer embossed shaping portion of the outer embossed shaping element and the fourth lateral embossed shaping portion being provided outwards with respect to the second outer embossed shaping portion of the outer embossed shaping element, the third and the fourth outer embossed portions being arranged on a respective side of the I-shaped portion of the central embossed shaping element.

12. The absorbent article according to claim 1, wherein the second absorbent structure comprises superabsorbent polymers in an amount of from 30 wt. %.

13. The absorbent article according to claim 1, wherein the second absorbent structure comprises a first side cut along the first longitudinal side edge and a second side cut along the second longitudinal side edge and wherein the first and the second side cuts are arranged in the front half of the absorbent core.

14. The absorbent article according to claim 1, wherein a ratio of a width at the narrowest section of the second absorbent layer at the first and the second side cuts and a width of the absorbent article at the same location is from 0.5:1 to 0.8:1.

15. The absorbent article according to claim 1, wherein the central embossed shaping element or the outer embossed shaping element comprising continuous or discontinuous embossment portions.
